(19) United States
(12) Patent Application Publication
(10) Pub. No.: US 2017/0027741 A1
(43) Pub. Date: Feb. 2, 2017

(54) METHOD AND APPARATUS FOR INDUCING MICTURITION

(71) Applicant: Lawrence Bruce, Andover, MA (US)

(72) Inventor: Lawrence Bruce, Andover, MA (US)

(21) Appl. No.: 15/179,172

(22) Filed: Jun. 10, 2016

Related U.S. Application Data
(60) Provisional application No. 62/197,897, filed on Jul. 28, 2015.

(51) Int. Cl. A61F 7/00; A61H 23/00; A61N 1/36

(52) U.S. Cl. CPC A61F 7/00; A61N 1/36014; A61H 23/00; A61F 2007/0018; A61F 2007/0047; A61F 2007/0048

(57) ABSTRACT

An apparatus to artificially induce micturition is presented. The apparatus includes a urination stimulation element in communication with a user, the urination simulation element providing a trigger to the user, said trigger for completing a parasympathetic feedback loop for initiation, reinitiation or continued flow of urine from the user. The apparatus further includes a receptacle, a handle in mechanical communication with the receptacle, an opening in the receptacle for receiving urination from a user, and a valve having a first side in fluid communication with the reservoir and having a second side in fluid communication with the receptacle.

### CROSS REFERENCE TO RELATED APPLICATIONS

The present application claims the benefit of U.S. Provisional Patent Application No. 62/197,897, filed on Jul. 28, 2015, which is incorporated herein by reference in its entirety.

### BACKGROUND

Micturition is the act of passing urine, also referred to herein as urination. The physiology of urination consists of both sympathetic and parasympathic nervous system aspects. The bladder has stretch receptors in the muscles of its walls that communicate via the pelvic nerve to the sacral spinal cord. When the bladder is full, the frequency of the nerve impulses increases. This triggers a signal to be sent to the Potine micturition center in the brain. The Potine micturition center communicates with the cerebral cortex about the appropriateness of urinating at this moment. If it is not, an inhibitory signal is sent via the sympathetic nervous system using the pudendal nerve to the external sphincter of the bladder to stay closed. If it is appropriate to urinate, the inhibitory signal is removed and the spinal cord communicates via the parasympathetic nervous system to contract the bladder and relax the external bladder sphincter to urinate.

There are many situations when the brain determines that it is inappropriate to urinate that causes discomfort and inconvenience. Paruresis (also known as shy bladder) is a condition wherein a person has trouble urinating when people are around. Many people are unable to urinate in a situation that is not very private such as a hospital or emergency room, or under pressure of time in a diagnostic lab to provide a urine sample. Many people are unable to void in a recumbent position. Many people are unable to urinate while being observed or in the presence of others in a public bathroom facility or other place in which the person does not feel the area is private. It can cause tremendous physical discomfort and embarrassment, and in extreme cases creates highly restricting effects on a person's life. Paruresis affects 21 million Americans, 220 million people world-wide. The traditional treatments for paruresis are psychotherapy, administration of diuretics, and catheterization.

### SUMMARY

There are known triggers that can induce urination. These triggers help to override the inhibitory signal that causes the bladder external sphincter to remain closed. Examples of triggers to complete the parasympathetic feedback loop required for initiation, reinitiation, and/or continued flow of urine include auditory triggers, tactile triggers, thermal triggers, or vibrational triggers. These triggers can be synthesized and integrated into urine collection vessels, handheld devices, or other means to facilitate urination in persons afflicted with paruresis.

It is an object of the present invention to provide a method that can be conveniently and perhaps discreetly used in order to achieve urination without any discomfort or pain. In particular, it is an object of the invention to provide a method capable of inducing urination without requiring any systemic administration of drugs or other medication, and also without requiring any invasive procedures.

It is also an object of this invention to provide a method for inducing urination that has absolutely no harmful side effects, and can safely be used on persons of any age.

Note that each of the different features, techniques, configurations, etc. discussed in this disclosure can be executed independently or in combination. Accordingly, the present invention can be embodied and viewed in many different ways. Also, note that this summary section herein does not specify every embodiment and/or incrementally novel aspect of the present disclosure or claimed invention. Instead, this summary only provides a preliminary discussion of different embodiments and corresponding points of novelty over conventional techniques. For additional details, elements, and/or possible perspectives (permutations) of the invention, the reader is directed to the Detailed Description section and corresponding figures of the present disclosure as further discussed below.

### BRIEF DESCRIPTION OF THE DRAWINGS

The foregoing will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention. The drawings are not necessarily to scale, emphasis instead being placed upon illustrating the principles of the invention.

FIG. 1 depicts a diagram of a first particular embodiment of an apparatus for inducing micturition in men in accordance with a particular embodiment of the present invention.

FIG. 2 depicts a diagram of a second particular embodiment of an apparatus for inducing micturition in men in accordance with a particular embodiment of the present invention.

FIG. 3 depicts a diagram of a first particular embodiment of an apparatus for inducing micturition in women in accordance with a particular embodiment of the present invention.

### DETAILED DESCRIPTION

The embodiments set forth below represent the necessary information to enable those skilled in the art to practice the invention and illustrate the best mode of practicing embodiments of the invention. Upon reading the following description in light of the accompanying figures, those skilled in the art will understand the concepts of the invention and recognize applications of these concepts not particularly addressed herein. It should be understood that these concepts and applications fall within the scope of the disclosure and the accompanying claims.

The preferred embodiment of the invention will now be described with reference to the accompanying drawings. The invention may, however, be embodied in many different forms and should not be construed as limited to the embodiment set forth herein; rather, this embodiment is provided so that this disclosure will be thorough and complete, and will fully convey the scope of the invention to those skilled in the art. The terminology used in the detailed description of the particular embodiment illustrated in the accompanying drawings is not intended to be limiting of the invention. In the drawings, like numbers refer to like elements.

This invention relates to a method and apparatus for triggering urination. Known sensory information, “triggers” to complete the parasympathetic feedback loop required to for initiation, reinitiating, and continued flow of urine include audible sounds, tactile sensations, including pouring a liquid on a location on the body, thermal sensations, to include warm or cold applied to various locations on the body, and vibrations applied to various locations on the outside of the body.

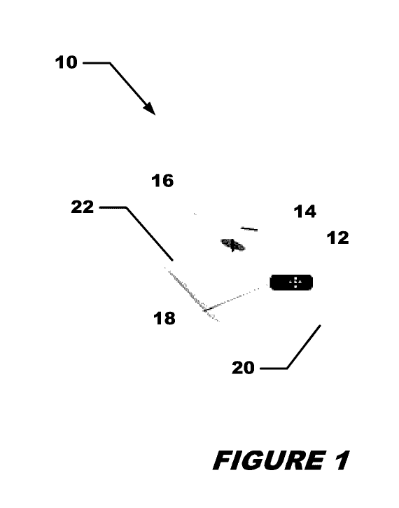

Referring to FIG. 1, one embodiment of the apparatus 10 for inducing micturition is shown. Apparatus 10 includes a receptacle 20 for receiving urination from a patient. Also shown is a particular embodiment of a urination stimulation element, in this instance comprising a handle 12 having a liquid reservoir, and a valve 16. As seen in FIG. 1, a liquid is held suspended and ready to dispense in a handle 12. A fill cap 14 is used to allow the handle 12 to be filled with liquid. The liquid is dispensed by opening the valve 16. The liquid flows upon the perineal area which may include the penis of an individual for the purpose of initiation, reinitiating, and/or continued flow of urine. In this particular embodiment the opening 22 into the receptacle is adapted for a male user. The sensation of water touching, running, and otherwise upon the penis of an individual may help to trigger the body's desired response of the initiation, reinitiating, and continued flow of urine. In one variation of this embodiment, a container is freestanding or attached to a receptacle to deposit urine and upon command dispenses a liquid upon selected areas of the body. This embodiment is designed for males.

In another embodiment the handle is not used as a liquid reservoir, instead a reservoir is attached to a receptacle to deposit urine and is also valve controlled. The liquid can be dispensed at varying rates using the valve.

In another embodiment, a mechanical vibratory device is used as a trigger and serves to provide a sensory signal to trigger the body's desired response of the initiation, reinitiating, and continued flow of urine.

In another embodiment, a low electrical voltage and amperage device is used as a trigger for urination. The electrodes may be applied to the sacral area of the spine or to the area of the symphysis pubis. The voltage and amperage can be modulated at varying frequencies to facilitate the urination response.

In another embodiment, a thermal device is used as a trigger for urination. The thermal device can be either warmer or cooler than body temperature and may be applied to the groin area or to the soles of the feet.

In another embodiment, auditory sensations are used as a trigger for urination. The audio sensations can be delivered by a solid-state audio playback module 18 that can be integrated into a urine receptacle, or be made to fit onto one as a separate module. The audio module can also be integrated into a child's toilet training device. The audio playback module can also be implemented as a stand-alone bodily-worn (in the ear) device that is very discreet.

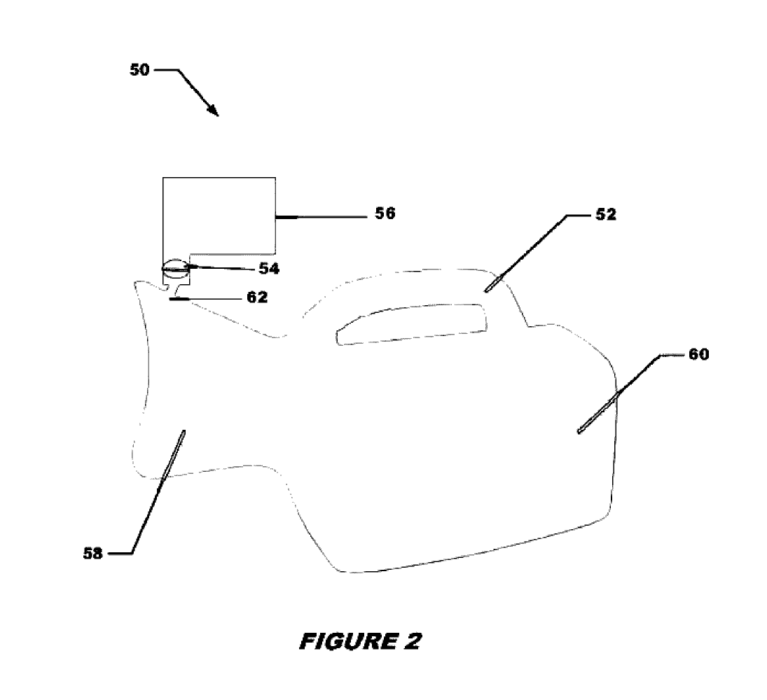

Referring now to FIG. 2 a second embodiment of the presently described apparatus 50 for inducing micturition is shown. Apparatus 50 includes a receptacle 60 for receiving urination from a patient. Apparatus 50 further includes a handle 52 in mechanical communication with the receptacle 60. Apparatus 50 further includes a liquid reservoir 56, and a valve 54, and a neck 62. As seen in FIG. 2, a liquid is held suspended in reservoir 56. The liquid is dispensed by opening the valve 54. The liquid flows upon the vulva and or perineal area of an individual for the purpose of initiation, reinitiating, and/or continued flow of urine. That the sensation of water touching, running, and otherwise upon the penis of an individual may help to trigger the body's desired response of the initiation, reinitiating, and continued flow of urine. In this embodiment the opening 58 is designed for females.

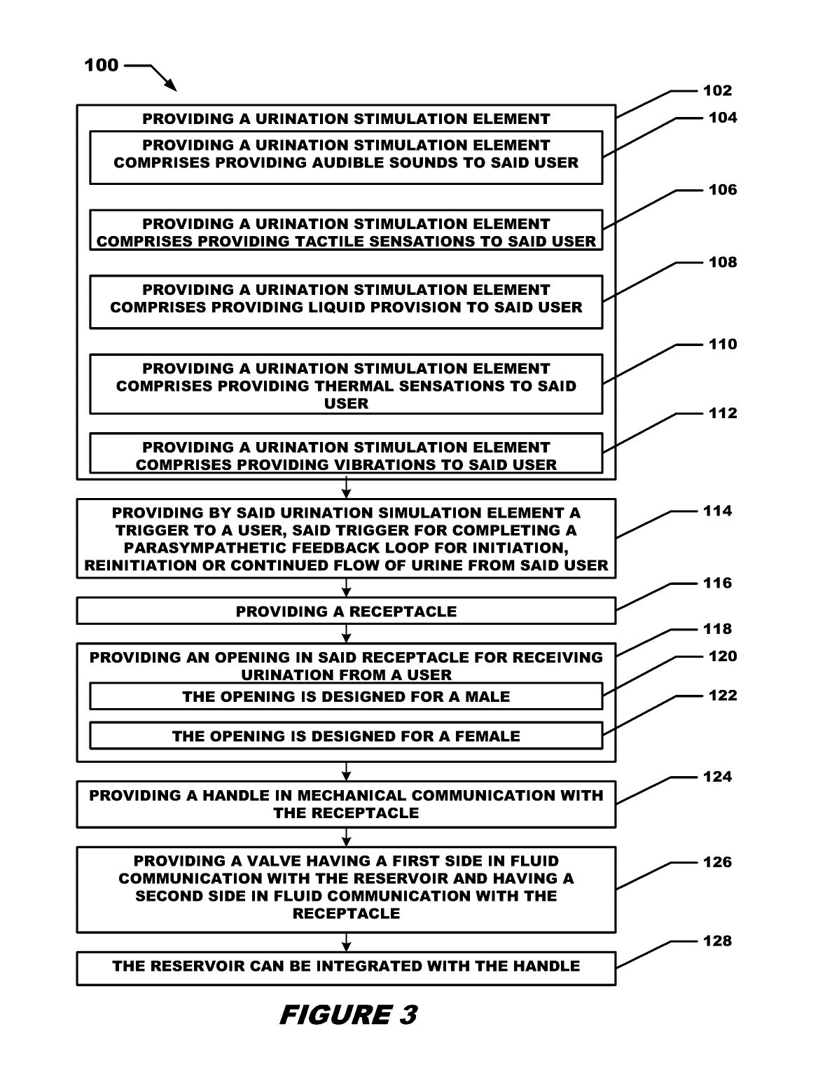

A flow chart of a particular embodiment of the presently disclosed method is depicted in FIG. 3. It will be appreciated by those of ordinary skill in the art that unless otherwise indicated herein, the particular sequence of steps described is illustrative only and can be varied without departing from the spirit of the invention. Thus, unless otherwise stated the steps described below are unordered meaning that, when possible, the steps can be performed in any convenient or desirable order.

The method 100 for artificially inducing micturition begins with processing block 102 which discloses providing a urination stimulation element. As shown in processing block 104 providing a urination stimulation element comprises providing audible sounds to said user. As shown in processing block 106 providing a urination stimulation element comprises providing tactile sensations to said user. As shown in processing block 108 providing a urination stimulation element comprises providing liquid provision to said user. Processing block 110 states wherein providing a urination stimulation element comprises providing thermal sensations to said user. Processing block 112 discloses wherein said providing a urination stimulation element comprises providing vibrations to said user.

Processing block 114 recites providing by said urination simulation element a trigger to a user, said trigger for completing a parasympathetic feedback loop for initiation, reinitiation or continued flow of urine from said user.

Processing block 116 discloses providing a receptacle. Processing block 118 recites providing an opening in said receptacle for receiving urination from a user. As shown in processing block 120, the opening is designed for a male, and alternatively, as shown in processing block 122, the opening is designed for a female.

Processing block 124 states providing a handle in mechanical communication with the receptacle. Processing block 126 discloses providing a valve having a first side in fluid communication with the reservoir and having a second side in fluid communication with the receptacle. As recited by processing block 128, in certain embodiments the reservoir can be integrated with the handle.

Throughout the entirety of the present disclosure, use of the articles “a” or “an” to modify a noun may be understood to be used for convenience and to include one, or more than one of the modified noun, unless otherwise specifically stated.

Elements, components, modules, and/or parts thereof that are described and/or otherwise portrayed through the figures to communicate with, be associated with, and/or be based on, something else, may be understood to so communicate, be associated with, and or be based on in a direct and/or indirect manner, unless otherwise stipulated herein.

Although the methods and systems have been described relative to a specific embodiment thereof, they are not so limited. Obviously many modifications and variations may become apparent in light of the above teachings. Many additional changes in the details, materials, and arrangement of parts, herein described and illustrated, may be made by those skilled in the art.

Having described preferred embodiments of the invention it will now become apparent to those of ordinary skill in the art that other embodiments incorporating these concepts may be used.

What is claimed is:

1. An apparatus to artificially induce micturition comprising:
a urination stimulation element in communication with a user, said urination simulation element providing a trigger to said user, said trigger for completing a parasympathetic feedback loop for initiation, reinitiation or continued flow of urine from said user.

2. The apparatus to artificially induce micturition further comprising:
a receptacle;
a handle in mechanical communication with said receptacle;
an opening in said receptacle for receiving urination from a user; and
a valve having a first side in fluid communication with said reservoir and having a second side in fluid communication with said receptacle.

3. The apparatus of claim 2 wherein said reservoir is integrated with said handle.

4. The apparatus of claim 1 wherein said urination stimulation element provides audible sounds to said user.

5. The apparatus of claim 1 wherein said urination stimulation element provides tactile sensations to said user.

6. The apparatus of claim 1 wherein said urination stimulation element provides liquid provision to said user.

7. The apparatus of claim 1 wherein said urination stimulation element provides thermal sensations to said user.

8. The apparatus of claim 1 wherein said urination stimulation element provides vibrations to said user.

9. The apparatus of claim 2 wherein the opening in the receptacle is shaped to receive a male user.

10. The apparatus of claim 2 wherein the opening in the receptacle is shaped to receive a female user.

11. A method for artificially inducing micturition comprising:
providing a urination stimulation element; and
providing by said urination simulation element providing a trigger to a user, said trigger for completing a parasympathetic feedback loop for initiation, reinitiation or continued flow of urine from said user.

12. The method of claim 11 further comprising:
providing a receptacle;
providing a handle in mechanical communication with said receptacle;
providing an opening in said receptacle for receiving urination from a user; and
providing a valve having a first side in fluid communication with said reservoir and having a second side in fluid communication with said receptacle.

13. The method of claim 11 wherein said reservoir is integrated with said handle.

14. The method of claim 11 wherein said providing a urination stimulation element comprises providing audible sounds to said user.

15. The method of claim 11 wherein said providing a urination stimulation element comprises providing tactile sensations to said user.

16. The method of claim 11 wherein said providing a urination stimulation element comprises providing liquid provision to said user.

17. The method of claim 11 wherein said providing a urination stimulation element comprises providing thermal sensations to said user.

18. The method of claim 12 wherein said providing a urination stimulation element comprises providing vibrations to said user.

19. The method of claim 12 wherein the opening in the receptacle is shaped to receive a male user.

20. The method of claim 12 wherein the opening in the receptacle is shaped to receive a female user.
